(19) United States
(12) Patent Application Publication
(10) Pub. No.: US 2006/0115529 A1
(43) Pub. Date: Jun. 1, 2006

(54) Fast-melting tablets having taste-masking and sustained release properties

(76) Inventors: Seonghoon Jeong, West Lafayette, IN (US); Susumu Kimura, Ichinomiya-shi (JP); Yourong Fu, West Lafayette, IN (US); Kinam Park, West Lafayette, IN (US)

Correspondence Address: JAMES H. MEADOWS AND MEDICUS ASSOCIATES, 2804 KENTUCKY, JOPLIN, MO, 64804, US

(21) Appl. No.: 11/267,568

(22) Filed: Nov. 4, 2005

Related U.S. Application Data
(63) Continuation-in-part of application No. 10/841,979, filed on May 7, 2004.
(60) Provisional application No. 60/624,959, filed on Nov. 4, 2004.
(60) Provisional application No. 60/468,449, filed on May 7, 2003.

(51) Int. Cl. A61K 9/20

(52) U.S. Cl. 424/464

(57) ABSTRACT

Fast-melting tablets contain particles of an active ingredient and ion-exchange resin complex to mask unpleasant taste associated with the active ingredient. The resin complex particles can be coated or uncoated to impart sustained release properties to the active ingredient. A fast-melting tablet also comprises a dry binder and bulk diluent to form highly plastic granules that are subsequently compressed into tablets.

### REFERENCE TO RELATED APPLICATION

This application claims the benefit of U.S. Provisional No. 60/624,959, filed Nov. 4, 2004, and is a continuation-in-part of U.S. Ser. No. 10/841,979, filed May 7, 2004, which claims the benefit of U.S. Provisional No. 60/468,449, filed May 7, 2003, the disclosures of which are incorporated herein by reference.

### FIELD OF THE INVENTION

The present invention relates to a fast-melting tablet for oral administration, which can release active pharmaceutical ingredients over a long period of time and/or can mask the unfavorable taste of the active ingredients.

### BACKGROUND OF THE INVENTION

For more than a decade, fast-melting tablet technologies have been steadily advancing in the development of patient-friendly dosage forms. The initial success of the first fast-melting tablet formulation initiated the development of different technologies. There are mainly three different technologies: freeze-drying, sublimation or heat molding, and direct compression. Fast-melting tablets are also known as a fast-disintegrating, fast-dispersing, rapid dissolving, rapid melting, and/or quick disintegrating tablets. The Food and Drug Administration (FDA) named all the approved fast-melting tablets as ‘orally disintegrating tablets’. The European Pharmacopeia used the name ‘orodispersible tablet’ for this kind of dosage forms.

The fast-melting tablet system is suitable for all age groups but it is especially useful for children, the elderly and schizophrenic patients who have difficulty in swallowing conventional tablets and capsules. Use of the fast-melting tablets can be extended to more general patients of daily medication regimens. This dosage form has all the advantages of solid dosage forms such as good stability, accurate dosing, small packaging size, and easy handling by patients. It also has the advantages of liquid formulations such as easy administration and minimal risk of suffocation resulting from physical obstruction by the dosage form.

Upon introduction into the mouth, fast-melting tablets dissolve or disintegrate quickly on the tongue in the absence of water for the easy administration of active pharmaceutical ingredients. However, fast disintegration can limit the number of the active ingredients that can be incorporated into the solid dosage form. For example, bad taste, short half-life, and instability in the gastric environment of the active ingredients are some of the challenges for this dosage form. Conventional methods of taste-masking or sustained drug delivery systems, like coating of the tablet matrix, cannot overcome these limitations due to the large size and weak disintegration on the tongue. Coating small drug particles is one of the possible alternatives; however, it may cause initial dose dumping or burst effect, if the coating is not complete and it ruptures after the manufacturing process.

Many active pharmaceutical ingredients are unpalatable or unattractive in their natural state. After a tablet disintegrates or dissolves in the saliva, the active ingredient in the tablet remains in the oral cavity until it is swallowed. Upon swallowing, the active ingredient can be absorbed through the membrane of gastrointestinal tract resulting in the desired therapeutic effect. It is estimated that there are about 10,000 taste buds on tongue, roof of the mouth, cheeks, and throat, and each bud has 60-100 receptor cells. These receptor cells interact with molecules dissolved in the saliva and produce a positive or negative taste sensation. Since fast-melting tablets dissolve or disintegrate in the patient's mouth, the active ingredients will be partially dissolved in close proximity to the taste buds. After swallowing, there should be minimal or no residue in the mouth. A pleasant taste inside the mouth becomes critical for patient compliance. Unless the active ingredient is tasteless or does not have undesirable taste, taste-masking techniques should be used.

Current taste masking is often achieved by a few methods, such as using sweet-tasting substances as diluents, adding flavors, or encapsulating the unpleasant drug into microparticles or granules. All of them have their own advantages and limitations together. An ideal taste masking technology should provide the active ingredient without grittiness and with good mouth feel. The amount of taste masking materials used in the dosage forms should be kept low to avoid excessive increase in tablet size. The taste masking technology should also be compatible with fast-melting tablet formulations. For example, if active ingredient particles are coated to minimize unpleasant taste, the coating should not be broken during compression or it should not be dissolved during wet granulation. Taste masking of bitter-tasting active ingredients is critical to the success of the fast-melting tablet formulations.

If an active pharmaceutical ingredient is unstable in low pH, it is very important to apply a method to circumvent the gastric environment, such as coating with enteric coating materials which are not dissolved in gastric pH. If not properly coated, most of the active ingredients will be chemically degraded in low pH, resulting in low bioavailability. Moreover, if the active ingredient does not have good pharmacokinetic properties, such as a short half-life, it is beneficial to apply a sustained release system to achieve more effective therapies reducing the side effects, to keep blood drug concentration levels within a therapeutic range, and to make administrations fewer, e.g., twice per day or even once per day, for better patient compliance. For many orally administered active ingredients, it is preferable that the ingredients be released in the body at a constant rate for a longer period of time, such as 12 hours or longer. This will improve the effectiveness of the active ingredients. Moreover, for better patient-compliance, after disintegration in the mouth, the device for the sustained release should be small so that patients cannot feel the unpleasant grittiness of bigger particles. The maximum particle size with which patients do not feel the sandy feeling is around 200 microns so the sustained and taste-masked systems should be less than that size.

There are many examples of different methods to modify the release profiles and/or to mask the bad taste of active pharmaceutical ingredients for oral drug administration. One of the methods that have been applied in the pharmaceutical industry is to change the active ingredients into a complex with ion-exchange resins for preparing drug/ion-exchange resin complexes (resin complexes). The advantages of ion-exchange resin in drug delivery devices can include a simple preparation method of drug/resin complex, no uncontrolled burst effect in the drug/resin complex even at high drug loading, and the exchanging capacity of the resin is the only limiting factor of drug loading. However, there are still some limitations using ion-exchange resin for drug delivery such as incomplete drug release when tightly crosslinked gel-type resins are used.

The term “ion-exchange” can be defined as an attractive electrostatic interaction of ions between a liquid (ionic active ingredient solution) and a solid phase (ion-exchange resin), without significant change in the structure and properties of the solid. Generally ion-exchange resins suitable for ion-exchange chromatography and deionization of water are good for this purpose. Therefore, the resulting resin complexes are a kind of salts formed between ionic pharmaceutically active ingredients and ion-exchange resins. For example, cation exchange resins form complexes with basic active ingredients and anion exchange resins form complexes with acidic active ingredients. When such active ingredient/resin complexes are administered through the gastrointestinal tract, the attached active ingredient molecules can be released by the ion-exchange reaction with counter ions in the stomach and the intestine. Such drug/ion-exchange resin complexes have been used for achieving the taste-masking and the sustained release properties mainly for liquid/suspension dosage forms.

Even though ion-exchange resins can be good active ingredient carriers for taste-masking and for controlled/sustained release to improve pharmacokinetic properties, they may not be sufficient alone to accomplish an improvement. In order to get better release properties and reduce the side effects of active ingredients further, coating or microencapsulation with various pharmaceutical materials using ion-exchange resin complexes as core materials can be applied. This method allows control of drug release by both ion-exchange resin and external encapsulation giving further control of the release rate and flexibility. This implies that the drug release rate can be controlled by one or a combination of diffusion resistance of the core (resin complex), diffusion resistance of the coating, and ion-exchange reaction rate, depending on the properties of the ion-exchange resins and coating materials used.

U.S. Pat. Nos. 4,221,778 and 4,847,077 (Raghunathan) propose making prolonged release pharmaceutical preparations containing a drug/ion-exchange resin complex, a substantial portion of which is treated with a solvating agent and provided with a water-permeable diffusion barrier coating. U.S. Pat. Nos. 4,859,461 and 4,859,462 (Chow et al.) propose enhancing coatability of sulfonic acid cation exchange resin particles with a high molecular weight polymer. U.S. Pat. No. 4,996,047 (Kelleher et al.) discloses an oral pharmaceutical composition containing sustained release drug/resin complexes, which are coated with a water-permeable diffusion barrier. U.S. Pat. No. 5,413,782 (Warchol et al.) proposes a drug/resin complex substantially free of carbon dioxide and/or bicarbonate. U.S. Pat. No. 6,001,392 (Wen et al.) proposes a mixture of coated and non-coated sulfonic acid cation exchange resins cross-linked with divinyl benzene onto which dextromethorphan has been loaded. U.S. Pat. No. 6,514,492 (Gao et al.) and U.S. Pat. No. 5,219,563 (Douglas et al.) propose formulations of oral liquid product using ion-exchange resins as carriers for eliminating the bitter taste of the active pharmaceutical ingredients, quinolones and ranitidine, respectively. U.S. Pat. No. 6,280,717 (Kamakura et al.) proposes a dry syrup preparation containing a cation exchange resin, a gelling agent, and a binder. U.S. Pat. No. 2005/0036977 (Gole et al.) proposes a taste-masking resinate containing a water-insoluble active substance complexed with ion-exchange resin.

U.S. Patent Publn. No. 2002/0146384 (Hughes et al.) proposes a resin/resinate combination for optimizing the release profile of active ingredients. U.S. Patent Publn. No. 2005/0112198 (Challapalli et al.) discloses a pharmaceutical formulation for the stabilization and sustained delivery of active pharmaceutical ingredients. U.S. Patent Publn. No. 2005/0181050 (Hirsh et al.) discloses a multi-particulate release composition for oral administration using coated drug/ion-exchange resin complexes in the form of small particles, which do not require impregnating agents to ensure the integrity of the release coating.

Most of the previous applications using uncoated and/or coated drug/resin complexes have been focused on suspension or liquid dosage forms. The main reasons for this may be poor compressibility of the resins and a change of release rate after manufacturing due to the granulation and compression processes. Compaction of the coated particles with suitable pharmaceutical excipients seems to provide a convenient method of formulating them into the tablet dosage forms. However, compaction of the coated or microencapsulated resin complex particles into tablets can have the problem of possible damage to the polymeric coating films, especially when the core-wall ratio is high where the polymeric wall is very thin. This problem is due to a mechanical stress the coated resin particles are subjected to during the compression procedure. Acceptable tablets containing coated resin particles should exhibit sufficient physical integrity to withstand handling and disintegrate rapidly into individual particles on the tongue. The size of the disintegrated particles should not be large enough to be felt by a patient, causing an unpleasant feeling like grittiness. The coated resin particles should not fuse into each other during compaction, and the drug release should not be affected by the compaction process. They may deform but should not rupture in order to maintain the initial release rate. In order to keep the release rate, the core of the coating should have some degree of plasticity, which can accommodate changes in shape and deformation during compression. With the above objects in mind, the present invention has been developed.

### SUMMARY OF THE INVENTION

The present invention is directed to a fast-melting tablet and method of making the same. A tablet contains a plurality of compressed highly plastic granules, wherein the granules contain an effective amount of particles of at least one active ingredient chemically complexed with an ion-exchange resin. A tablet also contains a dry binder and a bulk diluent. A tablet can also contain at least one coating substance for coating or microencapsulating the particles of active ingredient/ion-exchange resin complex in order to impart sustained release properties to the active ingredient.

In another aspect of the invention, a method of making a fast-melting tablet having taste-masking properties comprises providing a plurality of particles of an active ingredient/ion-exchange resin complex, combining a dry binder and a bulk diluent with the resin complex particles, treating the admixture with an aqueous wet granulation solution effective to form a wet mass of agglomerated particles, sieving and drying the agglomerated particles to isolate highly plastic granules, and compressing the granules under low pressure to afford the fast-melting tablet.

### DESCRIPTION OF THE DRAWINGS

FIG. 1 shows a scanning electron microscope (SEM) photo of Ethocel 100-coated dextromethorphan/Dowex® 50WX4-400 complexes prepared by a double emulsion-solvent evaporation method.

FIG. 2 shows SEM photos of Aquacoat® ECD-coated (Panel A) and Kollicoat SR® 30D-coated (Panel B) dextromethorphan/Dowex® 50WX4-400 complexes prepared in a fluidized bed.

FIG. 3 shows an in vitro drug release profile from a fast-melting tablet containing cetirizine/Amberlite IRP-64 complex in a simulated gastric fluid (pH=1.2) at 37° C.

### DETAILED DESCRIPTION OF THE INVENTION

The present invention is for a fast-melting (fast-disintegrating) tablet and method of making the same. As used herein, “fast-melting” is used synonymously with “fast-disintegrating”, although it should be understood that particles of the tablet can be micron-scale upon disintegration. As used herein, melting or disintegration of a tablet refers specifically to the same in the buccal cavity of a user, and therefore depends upon the user's saliva production to effect melting/disintegration.

A tablet of the invention comprises a pharmaceutically effective amount of at least one active ingredient, and at least one ion-exchange resin. An “effective amount”, as used herein, refers to an amount of the active ingredient that is capable of preventing, ameliorating, or curing a disease state or other physical or mental condition in a user, either in a single dose or in multiple doses. Typically, dosages received by the user are prescribed by a physician; however, individuals may be free to self-assess suitable doses in certain instances.

An ion-exchange resin of the invention is capable of binding ionically to an active ingredient so that an active ingredient/ion-exchange resin complex is formed. Many active ingredients are charged species and, therefore, lend themselves readily to ionic interactions with an ion-exchange resin. For instance, a negatively charged drug (perhaps one deprotonated by a base) is chemically attracted to and ionically bound to a positively-charged solid phase of an ion-exchange resin. Through this interaction, any negative counterion associated with the resin is displaced by the negatively charged drug to produce a drug/resin complex, also referred to herein as a “resinate”.

A tablet of the present invention is designed not only to melt rapidly in the buccal cavity, but also is designed to provide sustained release and/or taste-masking of an active ingredient. To this end, a tablet comprises highly plastic granules, which imbue the tablet with fast-melting properties. Additionally, the active ingredient/ion-exchange resin particles that comprise a tablet impart taste-masking and/or sustained release properties to the tablet.

Hence, a tablet of the invention comprises an active ingredient/ion-exchange resin complex, and at least one coating layer that envelopes the complex to control release of the active ingredient and/or to improve taste masking. Useful coating formulations involve polymeric ingredients as well as excipients conventionally employed in such coatings. It has been found that some coating polymers are resistant enough to keep the release rate unaltered after the manufacturing process. A coating material is layered onto an active ingredient/ion-exchange resin complex through a conventional process, such as fluidized bed coating, spray drying, hot-melt coating, pan coating, solvent evaporation, or coacervation.

Formulation of a tablet of the invention depends on the physical and pharmacological characteristics sought for the tablet. These can be summarized by the following objects: (1) Fast buccal disintegration without taste masking or sustained release, (2) fast buccal disintegration with taste masking but without sustained release, (3) fast buccal disintegration without taste masking but with sustained release, and (4) fast buccal disintegration with both taste masking and sustained release. In each of these cases, fast buccal disintegration is achieved primarily by formulating the tablet with a plastic material, a water penetration enhancing agent, and a binder so as to form highly plastic granules, which are compressed into the fast-melting tablets. This aspect of the invention is addressed more fully in U.S. Ser. No. 10/841,979, filed May 7, 2004. Taste masking is achieved in a tablet of the invention by complexing an active ingredient with an ion-exchange resin, so that an ill-tasting active ingredient is not significantly released into the buccal cavity free of the resin. Furthermore, sustained release properties are imparted to the active ingredient by suitably coating or microencapsulating the resin complexes, so that they are not substantially released or degraded until desired, e.g., in the small intestine.

A. Active Pharmaceutical Ingredients

An active ingredient suitable for use in this invention is any ionic active pharmaceutical ingredients (acidic, basic, or amphoteric). Preferably, the active ingredient is a free form of a basic pharmaceutical or a salt with a pharmaceutically allowed acid, prior to loading into an ion-exchange resin. Examples of active ingredients useful in the present invention include, but are not limited to, diphenhydramine hydrochloride, cetirizine hydrochloride, dextromethorphan hydrobromide, venlafaxine hydrochloride. These drugs are practically all water soluble and ionizable. Other water-soluble pharmaceuticals or drugs may be used in this invention. Nutritional compounds and other chemical compounds can be used. Examples of nutritional compounds that may be used with this invention include vitamins, minerals and dietary supplements.

B. Ion-Exchange Resins and Complex Formation

An ion-exchange resin suitable for use in the present invention should be water-insoluble and not absorbed in the body without any unfavorable effects. It should be composed of a pharmacologically inert matrix containing covalently bound functional groups that can be ionized under certain pH conditions. The ionic functional groups are —SO₃⁻ and —COO⁻ in cation exchange resins and —N⁺— in anion exchange resins. There are two types of ion-exchange matrices: an organic matrix, which is synthetic such as polymers or copolymers of methacrylic acid, acrylic acid, sulfonated styrene, sulfonated divinylbenzene, or partially synthetic such as modified cellulose and dextrans; and an inorganic matrix, which can be silica gel modified by the addition of ionic groups. The ionic groups can be strongly acidic (sulfonic acid), weakly acidic (carboxylic acid), strongly basic (quaternary ammonium), weakly basic (primary amine), or a combination of acidic and basic groups.

Polymeric ion-exchange resins are generally synthesized through a crosslinking reaction with suitable vinyl monomers. Crosslinking agents can be divinyl or polyvinyl compound, and divinylbenzene (DVB) is the most common one. For example, sulfonic polystyrene resins are crosslinked with styrene and DVB to which the sulfonic acid groups are attached by treatment with sulfuric acid. Anionic exchange resins can be prepared using the same process. However, instead of sulfonation with sulfuric acid, the crosslinked polystyrene is chloromethylated and then treated with a tertiary amine to get a quaternary amine binding site. Weak cation exchange resins, such as those based on carboxylic acid, are prepared by the crosslinking polymerization of carboxylic acids (methacrylic acid or acrylic acid) with DVB. The total capacity of an ion exchange resin is generally defined as the total number of available chemical equivalents for exchange per unit weight or unit volume of the resin. The capacity can be expressed as milliequivalents per gram of resin (meq/g) or milliequivalents per milliliter of resin (meq/ml). If a resin is highly crosslinked, it would be difficult to introduce additional functional groups. Sulfonation is accomplished after the crosslinking has been completed. During the sulfonation, the sulfonic acid groups are introduced both inside and surface of the resin particle. Similarly, the quaternary ammonium groups are introduced after the polymerization has been completed and they are introduced inside the particle as well as its surface. When they are highly crosslinked, fewer functional groups can be introduced inside the particles resulting in a slightly reduced total capacity of the resin. However, if a wet volume basis is used to measure the capacity of a resin, it is a different story. Even though fewer functional groups are introduced into the highly crosslinked resins, these groups are spaced closer together on a volume basis because the volume of water is reduced as the crosslinking increases. Therefore, the capacity on a wet volume basis increases as crosslinking increases.

Ion-exchange resins can swell due to the substitution of ionic groups and the extent of swelling is dependent on the degree of substitution of the ionic groups and the degree of crosslinking. They can be divided into gel or microporous based on their morphological structure. The gel-type resins are prepared by the suspension polymerization of styrene or carboxylic acids with a crosslinking agent. The resin is usually crosslinked from 2 to 20%. Since swelling of resins may cause some limitations of the ion-exchange resin process, the polymer beads are usually crosslinked highly to minimize the swelling, which results in slow diffusion of ions. Microporous resin structure can be prepared by adding porogen during the polymerization process, which is removed after the polymerization. This structure will make the ion exchange fast.

The pKa of ion-exchange resins with sulfonic acid is around 1 and that of carboxylic acid is between 4 and 6. Therefore, carboxylic acid functional groups have poor dissociation in an acidic medium. The resins of carboxylic acid have little complex formation with salt forms of drugs other than HCl salt. The size of the resins is preferably about 10 to about 400 μm, more preferably 20 to 300 μm, in diameter. Particle sizes below the lower limit are hard to handle during the manufacturing processes and particle sizes above the upper limit are gritty on the tongue when incorporated into fast-melting tablets, chewing tablets, or suspensions, and also tend to fracture when subjected to drying/hydrating cycles.

Examples of ion-exchange resins that may be used with this invention include Amberlite® IRP-64 (weakly acidic), Amberlite® IRP-69 (strongly acidic), Amberlite® IRP-88 (weakly acidic), Duolite™ AP-143 (strongly basic), Dowex® 50WX series (strongly acidic) and Dowex® 1X series (strongly basic). One of the most common resins is Amberlite® IRP-69 (Rohm and Haas), which is sulfonated polymers composed of polystyrene crosslinked with 8% of divinylbenzene, with an ion exchange capacity of about 4.5 to 5.5 meq/g of dry resin. It consists of irregularly shaped particles with a size range of 47 to 149 μm, produced by milling large particles.

Another most common resin is Dowex® 50WX series (Dow Chemical Company). There are mainly four products with different particle size distribution: cut-off mesh size is U.S. Sieve No. 50 (300 μm) in the case of Dowex® 50WX2-50, 100 (150 μm) in Dowex® 50WX2-100, 200 (75 μm) in Dowex® 50WX2-200, and 400 (38 μm) in Dowex® 50WX2-400. Crosslinking is another important factor, which can influence physical properties, equilibrium conditions, drug loading, and drug release profiles. Resins of various degrees of permeability are dependent on the divinylbenzene content, which was described as the degree of resin crosslinkage and the number after X is the percentage of divinylbenzene in the resin polymer. For example, Dowex® 50WX2-50 contains 2% divinylbenzene with particle size is bigger than 50 mesh. Total exchange capacity of 2, 4 and 8% crosslinkage resins are 0.6, 1.1 and 1.7 meq/ml, respectively. Any ion exchange resin that is useful for taste masking and sustained release applications with active ingredients can be used in this invention. Furthermore, combinations of multiple kinds of ion-exchange resins are also applicable for this invention.

Active pharmaceutical ingredients can be loaded into ion-exchange resins by exposing the resin to active ingredient solution. There are mainly two methods: batch and continuous (column) processes. For the batch process, the ion-exchange resin particles can be dispersed in active ingredient solution with specific drug/resin ratio under mixing at room or elevated temperature. For the continuous process, any glass liquid chromatography column or like can be used. A certain amount of the ion-exchange resins is slurried with water and transferred to the column. Sometimes, to stabilize the packing, the resin is backwashed with water using a peristaltic pump and then drug solution is pumped up-flow or down-flow. The complex formation occurs at room or elevated temperature. The ending point of the active ingredient loading can be determined by analyzing the active ingredient concentration of the effluent or by simply measuring the pH changes.

If the batch method is used for active ingredients loading, ion-exchange by-products will be produced, thereby changing the pH or ionic environments in the reaction medium. This will decrease the equilibrium rate and loading efficiency. For example, in the case of a cation exchange resin with the functional group SO₃⁻H⁺, as complex formation proceeds, acidic by-products are produced. They will change the pH of the reaction medium and compete with the counter ionic active ingredients in the bulk solution. To overcome this limitation and increase the loading efficiency, this invention uses a different batch method, called ‘a modified batch method’ depending on the applicability. After the batch process, the supernatant is decanted and fresh drug solution is added. This will remove competing ions (by-products) from the first batch. The same process can be followed until most of the functional groups are occupied depending on the loading efficiency and physicochemical properties of the ion-exchange resins. The complex is separated from the supernatant by vacuum filtration, washed with deionized water to remove any uncomplexed drug, and then dried. It is found that the ratio between the loaded active pharmaceutical ingredient and resin is from 0.5:1 to 3:1, depending on the properties of active ingredients and ion-exchange resins.

C. Coating Materials and Their Properties

It may not be sufficient to obtain the desired taste-masking and sustained release properties only with an ion-exchange resin, even though they are still good pharmaceutical excipients or matrices for that purpose. In order to get better release properties and reduce the side effects of the active pharmaceutical ingredients further, coating or microencapsulation with various pharmaceutical materials can be applied using ion-exchange resin complexes as core materials. This method can allow control of drug release by both ion-exchange resin and external encapsulation giving further flexibility on drug release rate control. This implies that the drug release rate can be controlled by one or more combinations of diffusion resistance of the core (resin complex), diffusion resistance of the coating, and ion-exchange reaction rate depending on the properties of used ion-exchange resins and coating materials.

Any coating procedures can be used as long as they can provide a continuous and reproducible coating on each particle of drug/resin complexes. In the following illustrative examples, a solvent evaporation microencapsulation method and fluid-bed coating apparatus having the Wurster configuration are applied. Additionally, waxes can be used as a coating material. Generally, they have been used widely as one of the most common ingredients in tablet or capsule formulations to provide a lubricating and/or gliding effect. However, when incorporated in a tablet matrix, they can show extended drug release properties. Hot-melt coating is a good example to provide sustained release and/or taste-masking effects of the waxes. For wax-coating, many waxes, including glyceryl behenate (Compritol 888 ATO®), polyethylene glycols (PEGs), stearic acid, glyceryl monostearate, and hydrogenated vegetable oils can be included.

The coating materials can be any of a large number of natural or synthetic film forming materials which can be used alone, mixed with each other, and/or mixed with plasticizers, pigments and/or other substances to modify the characteristics and to improve the appearance of the coating. Major components of the coating should be water insoluble but permeable and affording a diffusion barrier. However, it might be desirable to incorporate a water-soluble substance, such as methylcellulose, to modify the permeability of the coating, or to incorporate an acid-insoluble, base-soluble substance to act as an enteric coating. The coating materials may be applied as dispersion in an aqueous fluid or as solution in organic solvents. Useful coating materials for taste masking can include, but are not limited to, methacrylate polymers, cellulosic polymers, and polysaccharides. Ethylcellulose (EC), hydroxypropyl cellulose (HPC), hydroxypropylmethyl cellulose (HPMC), and methacrylate polymers, such as Eudragit E, Eudragit R S, Eudragit R L, Eudragit N E, are preferred.

Polysaccharides, such as maltodextrin, can be used as a coating in this invention. Mixtures of these materials also can be applicable for the coating. Sustained release coatings generally include, but are not limited to, ethylcellulose (EC), hydroxypropyl cellulose (HPC), hydroxypropylmethyl cellulose (HPMC), hydroxypropyl-methylcellulose phthalate (HPMCP), cellulose acetate phthalate (CAP), polyvinyl acetate (PVA), and methacrylate polymers, such as Eudragit L, Eudragit R S, Eudragit R L, Eudragit N E. Any mixtures of them are applicable, too. Many of the polymers are formulated into aqueous colloidal dispersions as latexes or pseudo-latexes to prevent environmental hazards due to the use of organic solvents.

There are many aqueous EC latex dispersions and Aquacoat® and Surelease® are typical examples. Aquacoat® is the aqueous EC pseudolatex stabilized with sodium lauryl sulfate and cetyl alcohol. Surelease® is prepared by a phase inversion emulsification method and contains ammonium oleate as a stabilizer and dibutyl sebacate as a plasticizer. With EC dispersions, plasticizers are required to reduce the minimum film formation temperature (MFT) below the coating temperature and to enhance the coalescence process. However, during the drying of Surelease®, ammonia evaporates leaving oleic acid as a plasticizer within the film so Surelease® may not need any plasticizers unlikely to Aquacoat®. More preferably, Kollicoat® SR 30D (polyvinyl acetate), manufactured by BASF Corporation, can be used for the coating. Kollicoat® SR 30D is an aqueous colloidal poly(vinyl acetate) dispersion and has low MFT, which is around 18° C. so it does not require plasticizer addition or post-thermal treatment (curing) after coating. Similar to other polymer dispersions, the rate of drug release can be adjusted by changing the coating level. Its release rate is independent of the pH and ionic strength of release medium.

Before the fluid-bed coating, impregnating agents can be treated onto the resin complex particles to prevent the rupture of the coating due to the swelling of the resin particles. Since the final dosage forms are mainly suspension/liquid preparations, the coated resin complexes meet aqueous environment as soon as they are formulated into the dosage form. If the coating materials are not strong enough to overcome the swelling of the core resin complexes, they will rupture resulting in significantly changed the release profiles. However, this invention requires no impregnating agents because coating materials with more mechanical strength are used and the main application of the coated resin particles is fast-melting tablets. There is a low possibility of meeting aqueous environment until the tablets are administered so there is limited swelling of the core resin complexes during storage. Moreover, the mechanical strength of the coating is quite resistant to swelling and to manufacturing process. For example, the films of Kollicoat® SR 30D polymer without plasticizers are slightly brittle in a dry state. However, when wet, they are flexible enough to be elongated more than 100% so the crack formation on the surface of coating due to the swelling of the core can be prevented. Moreover, a small amount of plasticizer can increase the flexibility of the polymer significantly. In this example, 5% of triethyl citrate is used, and the elongation at break value is more than 250%.

Final dosage form will affect the strategy of formulation development. Water-soluble plasticizers may not be used for liquid preparation because they tend to be leached out during the storage resulting in the formation of small pores or channels. It may change the drug release profiles compared to the initial ones. However, in the case of solid dosage forms, it can be prevented. Therefore, plasticizers can be added into the coating so that the mechanical strength of the polymer films can be modified depending on the properties of the polymers and plasticizers.

Since the final dosage form is a fast-melting tablet, the coated particles are compressed with suitable tablet excipients. There are generally two issues when the coated resin particles are compressed into the tablet. One is the effect of types and coating level of the polymer and the other is the effect of excipients. In fact, if not compressed, the mechanical properties of the coating are not so critical because burst effect or loss of sustained release might not be possible. For example, the coated particles can be filled into hard gelatin capsules. Therefore, the two issues have to be considered carefully for the development of successful taste-masked and/or sustained release fast-melting dosage forms. This invention shows there is almost no change in drug release profiles before and after granulation and compression. When investigated through scanning electron microscopy, no cracks are observed showing good mechanical properties of the coating polymers in this invention.

The compressed fast-melting tablets containing coated resin particles should disintegrate quickly into individual particles in the oral cavity. The coated particles should not fuse into a matrix during and after compression. Moreover, the drug release rate should not be affected by the compaction process. It is acceptable for the coating to deform but there should be no rupture. The coating films are preferably elastic so that there will be no rupture during compression. Therefore, the mechanical properties of the coating films are important for successful formulation development. Besides the permeability of the coating polymers governing the release rate, the mechanical properties of the coating are considered to examine their suitability for the coating of resin particles to be compressed into tablet dosage forms.

Preferred coating substances are selected from vinyl polymers, (meth)acrylate polymers, cellulosic polymers, waxes, polysaccharides, and mixtures thereof. Among the vinyl polymers are polyvinyl acetate and polyvinyl alcohol-polyethylene glycol. Preferred cellulosic polymers are ethylcellulose (EC), hydroxypropyl cellulose (HPC), hydroxypropylmethyl cellulose (HPMC), hydroxypropyl-methylcellulose phthalate (HPMCP), and cellulose acetate phthalate (CAP). Preferred (meth)acrylate polymers are aminoalkyl methacrylate copolymers, ammonioalkyl methacrylate copolymers, methacrylate copolymers, ethyl acrylate-methyl methacrylate coppolymer, metacrylic acid-ethyl acrylate copolymer, and mixtures thereof. A wax is preferably selected from glyceryl behenate, polyethylene glycols, stearic acid, glyceryl monostearate, hydrogenated vegetable oils, and mixtures thereof. A preferred polysaccharide is maltodextrin.

D. Formulation of Fast-Melting Tablet

A fast-melting tablet is formed by combining coated or uncoated active ingredient/ion-exchange resin complex particles with a dry binder and a bulk diluent to form an admixture thereof. The admixture is treated with a wet binder (aqueous granulation solution) effective to form a wet mass of agglomerated particles. The agglomerated particles are then sieved and dried, and compressed under low pressure to afford the fast-melting tablet. It should be noted that a single chemical component can serve as a dry binder, bulk diluent, and/or wet binder. A fast-melting tablet can be formed utilizing a conventional granulating machine.

Any materials, usually polymers, having high binding properties in the powder state can be used as a dry binder. Representative dry binders that can be used in making the granules include, but are not limited to, maltodextrin, dextrin, ethylcellulose, polymethacrylates, pregelatinated starch (e.g., LYCATAB® C by Roquette American Inc.). Maltodextrin can be obtained commercially, and examples are MALTRIN series (maltodextrins and corn syrup solids forms by Grain Processing Corp.), MALTRIN QD series (maltodextrins and corn syrup solids quick-dispersing forms by Grain Processing Corp.), and GLUCIDEX® IT (maltodextrins and spray-dried glucose syrups by Roquette American Inc.). MALTRIN QD series are used preferably because they are made to have high porosity inside the agglomerates in addition to their excellent binding property.

A bulk diluent is provided for a certain volume and weight in a tablet. To be used in making fast-melting tablets, a bulk diluent should be highly water-soluble but should at least be highly dispersible. Common diluents are highly water-soluble carbohydrates. Any types of carbohydrates can be used in the formulations described in this invention. Examples are dextrates, dextrin, dextrose, fructose, lactitol, lactose, maltitol, maltose, mannitol, sorbitol, sucrose, erythritol, and xylitol. Those diluents that are less water-soluble but highly dispersible include microcrystalline cellulose, silicified microcrystalline cellulose, powdered cellulose, cellulose acetate, calcium sulfate, calcium carbonate, dibasic calcium phosphate, tribasic calcium phosphate, and carboxymethylcellulose-calcium salt. Various combinations of carbohydrates and polymers can also be used. Examples include STARLAC® (spray-dried solid containing 15% maize starch and 85% alpha-lactose monohydrate from Roquette American, Inc.), MICROCELAC® (spray-dried solid containing 75% alpha-lactose monohydrate and 25% microcrystalline cellulose from Meggle excipients & technology), and CELLACTOSE® (spray-dried compound consisting of 75% alpha-lactose monohydrate and 25% cellulose powder by Meggle excipients & technology). A preferred grade of the material used as bulk diluent is the direct compressible grade. Materials prepared with high porosity, e.g., by spray drying, are even more preferred. Examples of porous bulk diluents are STARLAC®, MICROCELAC®, CELLACTOSE®, MANNOGEM EZ spray (spray dried mannitol from SPI Pharma, Inc.).

Solutions for wet granulation are made of highly concentrated carbohydrates or polymers with high water solubility. After the drying of wet granules, the solidified solute can dissolve quickly upon contact with water. The type and quantity of solute in solutions for the wet granulation can be adjusted in relation to the dry binders to give the granules desirable physical properties, such as compressibility and good binding properties. One example of effective solutions for wet granulation is 70% sorbitol solution. Other possible materials include acacia, alginic acid, CARBOMER, carboxymethylcellulose, cellulose, dextrin, gelatin, hydroxyethylcellulose, hydroxypropylcellulose, hydroxypropyl methylcellulose, methylcellulose, polydextrose, poly(ethylene oxide), povidone, and sodium alginate.

In the present invention, granules are preferably prepared by wet granulation, but the method of making the granules is not limited to wet granulation. After the coated/uncoated resin complex, dry binder, diluent, and other components are mixed, a solution for the wet granulation is added gradually while the dry materials are continuously stirred until a wet mass with desirable properties is obtained. The resultant wet mass is screened through a sieve with a desired particle size and then dried. The dried granules may be further combined with superdisintegrant, superporous hydrogel particles, effervescent agents, lubricants, flavoring agents, or coloring agents in a blender before compression. Lubricant can be dry-sprayed onto tablet tooling during compressing.

### E. CONCLUSION

It has been discovered that when highly plastic granules are used to incorporate coated and/or uncoated resin complex particles and compressed at low pressures, fast-melting tablets can be obtained that exhibit good drug release profiles of the resin particles. The highly plastic granules are composed of a dry binder and a bulk diluent, in addition to the resin complex particles, and are prepared by utilizing common granulating and compression machines. The granules act like a cushion for the complex particles in the tablet matrix. Moreover, they can protect the coated drug/resin complex particles from rupturing the coating during the manufacturing process. The tablet of this invention may contain other optional ingredients to improve patient compliance and to provide pleasant looking final products, for example, natural or artificial sweetener, flavoring agents, and colorants. When a tablet of the present invention is placed into the oral cavity, and particularly on the tongue, it melts (i.e., disintegrates) in less than thirty seconds, and sometimes shorter times, depending on the movement of the tongue. Compositions and methods related to the formation of highly plastic granules and fast dissolving tablets are disclosed in U.S. Ser. No. 10/841,979, filed May 7, 2004, the disclosure of which is incorporated herein by reference.

The present invention is now described by way of the following examples, which illustrate but do not limit it.

### EXAMPLES

### Example 1

### Dextromethorphan/Amberlite® IRP-69 Resin Complex

The dextromethorphan/Amberlite® IRP-69 complex was prepared by a batch method. The purified Amberlite® IRP-69 resin particles were sieved using Rotap RX-29 (Mentor, Ohio) to divide the particles into 106˜150 μm, 75˜106 μm, and <75 μm size particles. The particles of each part (38 g) were dispersed in 1.9 w/v % of the drug solution (2000 ml) under magnetic stirring at room temperature for 24 hours. The drug-loaded ion-exchange resin was separated from the supernatant by vacuum filtration, washed with de-ionized water to remove any uncomplexed drugs, and then dried in an oven at 45° C. The amount of loaded drug was 46˜50 mg in 100 mg dextromethorphan/Amberlite® IRP-69 complex particles.

| Ingredient | Amount | |
|---|---|---|
| Dextromethorphan HBr monohydrate | 38 | g |
| DI water | 2000 | ml |
| Amberlite ® IRP-69 | 38 | g |

### Example 2

### Dextromethorphan/Dowex® 50WX Resin Complex

The dextromethorphan/Dowex® 50WX resin complexes were prepared by batch, modified batch, and a continuous method. For the batch method, the previously purified Dowex® 50WX2-400 resin particles (38 g dry weight) were dispersed in a 1.9 w/v % of the drug solution (2000 ml) under magnetic stirring at room temperature for 5 hours. For the modified batch method, after decanting the clear supernatant of the above batch carefully, another 2000 ml of the fresh drug solution was added and stirred again for 5 hours at room temperature. The drug/ion-exchange resin complexes were separated from the supernatant by vacuum filtration, washed with de-ionized water to remove any uncomplexed drug, and then dried in the oven at 45° C. The amount of loaded drug was 52 mg for the batch method and 72 mg for the modified batch method in 100 mg dextromethorphan/Dowex® 50WX2-400 complex. When the modified batch method was used, the amount of loaded drug was 63 mg in 100 mg Dowex® 50WX4-100 resin complex, 65 mg in 100 mg Dowex® 50WX4-200 resin complex, 69 mg in 100 mg Dowex® 50WX4-400 resin complex, 54 mg in 100 mg Dowex® 50WX4-50 resin complex, and 46 mg in Dowex® 50WX8-400 resin complex.

For the continuous method, a luer-lock and non-jacketed glass liquid chromatography column (size: 1.0 cm×20 cm, bed volume 16 ml, Sigma-Aldrich) was used. A specific amount of the Dowex® WX2-400 resin was slurried with water and transferred to the glass column equipped with a coarse fritted-glass disk at the bottom. To stabilize the packing, the resin was backwashed with water using a peristaltic pump and then 1.9% drug solution was pumped up-flow at a rate of 70 ml/h. The complex formation occurred at room temperature. The drug/ion-exchange resin complex was collected by vacuum filtration, washed with de-ionized water to remove any uncomplexed drug, and then dried in an oven. The amount of loaded drug was 72 mg in 100 mg dextromethorphan/Dowex® 50WX2-400 complex.

### Example 3

### Release of Dextromethorphan from Different Particle Sizes of Amberlite® IRP-69 Resin Complexes

A drug release test from uncoated Amberlite® IRP-69 resin complex particles was conducted according to the USP 27 Apparatus 2 guidelines (paddle method) (Vankel® VK 7000, Vankel, Edison, N.J.) with 900 ml dissolution medium maintained at 37±0.5° C. and mixed at 100 rpm. The dissolution media used in this study were 0.1N HCl (pH=1.1˜1.2). Samples were withdrawn at predetermined time intervals and analyzed for drug content using HPLC system (Agilent 1100 Series, Agilent Technologies, Waldbronn, Germany) at a wavelength of 280 nm. The following release data were obtained showing that uncoated resin complex does not have significant sustained release properties. However, the release profiles are significantly dependent on the size distribution of the particles.

| | Cumulative dextromethorphan | | |
|---|---|---|---|
| | released (mg) from | | |
| | uncoated Amberlite IRP-69 complex | | |
| Time (hours) | <75 μm | 75˜106 μm | 106˜150 μm |
| 0.25 | 43 | 19 | 14 |
| 0.5 | 45 | 28 | 20 |
| 1 | 47 | 37 | 29 |
| 2 | 47 | 44 | 38 |
| 3 | 47 | 47 | 42 |
| 4 | 47 | 48 | 44 |
| 6 | 47 | 48 | 45 |
| 8 | 47 | 48 | 46 |
| 12 | 48 | 48 | 46 |

### Example 4

### Release of Dextromethorphan from Different Particle Sizes of Uncoated Dowex® 50WX Resin Complexes

A drug release test from uncoated Dowex® 50WX resin complex particles was conducted as described for Example 3. The following release data were obtained showing that the release profiles are significantly dependent on the size distribution of the resin particles.

| | Cumulative dextromethorphan | | | |
|---|---|---|---|---|
| | released (mg) from uncoated | | | |
| | Dowex ® 50WX complexes | | | |
| Time (hours) | 50WX4-50 | 50WX4-100 | 50WX4-200 | 50WX4-400 |
| 0.08 | 0 | 5 | 5 | 11 |
| 0.25 | 3 | 9 | 12 | 23 |
| 0.5 | 6 | 16 | 20 | 33 |
| 1 | 10 | 26 | 31 | 42 |
| 2 | 16 | 38 | 41 | 46 |
| 3 | 23 | 43 | 45 | 49 |
| 4 | 26 | 45 | 47 | 49 |
| 6 | 27 | 47 | 48 | 50 |
| 8 | 28 | 48 | 49 | 50 |
| 24 | 29 | 50 | 51 | 51 |

### Example 5

### Release of Dextromethorphan from Dowex® 50WX Resin Complexes at Different Crosslinking Ratios

A drug release test from uncoated Dowex® 50WX resin complex particles was conducted as described for Example 3. The following release data show that the release profiles are significantly dependent on the crosslinking ratio of the resin particles.

| | Cumulative dextromethorphan | | |
|---|---|---|---|
| | released (mg) from | | |
| | uncoated Dowex ® 50WX complexes | | |
| Time (hours) | 50WX2-400 | 50WX4-400 | 50WX8-400 |
| 0.08 | 26 | 11 | 6 |
| 0.25 | 38 | 23 | 13 |
| 0.5 | 44 | 33 | 19 |
| 1 | 48 | 42 | 27 |
| 2 | 49 | 46 | 36 |
| 3 | 49 | 49 | 38 |
| 4 | 49 | 49 | 42 |
| 6 | 50 | 50 | 45 |
| 8 | 50 | 50 | 47 |
| 24 | 50 | 51 | 47 |

### Example 6

### Ethylcellulose-Coated Dextromethorphan/Dowex® 50WX4-400 Resin Complexes by Microencapsulation Method

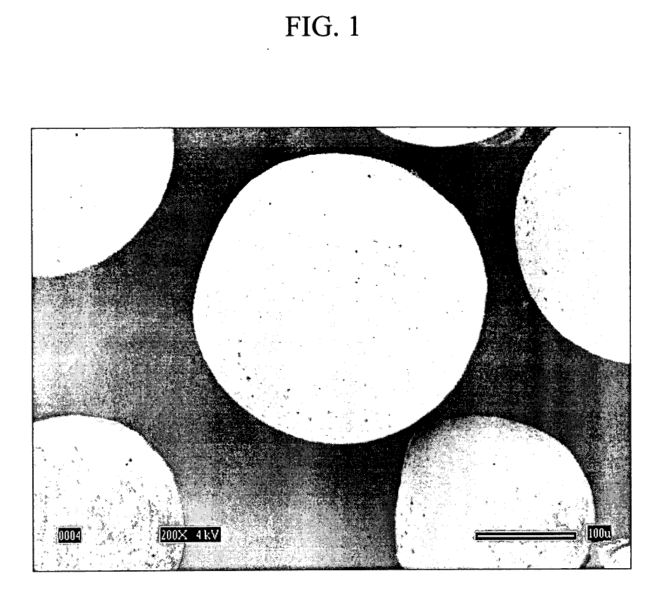

Ethylcellulose-coated dextromethorphan/Dowex® 50WX4-400 complex particles were prepared using a water in oil in water (W/O/W) double emulsion-solvent evaporation method. Ten grams of dextromethorphan/Dowex® 50WX4-400 complex were poured into 200 ml of methylene chloride containing 3.5% ethylcellulose (EC) polymers (Ethocel 20, 45, and 100; Dow Chemical Company). The solution was mixed for 30 sec using a vortex mixer. To make the water in oil (W/O) emulsion, 10 ml of water was added to the organic phase and mixed again with the vortex mixer for 1 min. The resulting W/O emulsion was poured under stirring into 8000 ml of aqueous 0.5% polyvinyl alcohol (PVA) solution to form a W/O/W double emulsion and continuously stirred for 3 hours at room temperature until most of the methylene chloride evaporated, leaving solid capsules. The microcapsules were collected by filtration, washed with distilled water at least three times, and dried in a 45° C. oven for 24 hours. The dried microcapsules were sieved using US standard test sieve No. 35 (500 μm) only to deaggregate the particles. A scanning electron microscope (SEM) photograph of the particles is shown in FIG. 1 and the average particle size was around 200 μm.

### Example 7

### Release of Dextromethorphan from Ethylcellulose-Coated Resin Complex Particles

A drug release study from the EC-coated dextromethorphan resin complex particles was conducted as described above. The release profiles were determined in terms of different types of Ethocel and relative amounts of drug and resin complex. The following release data were obtained showing that coating has significant sustained release properties and it depends on the different polymers and different extent of coating level.

| | Cumulative dextromethorphan | | | |
|---|---|---|---|---|
| | released (mg) from uncoated | | | |
| | and various EC-coated resin complexes | | | |
| Time (hours) | No coating | Ethocel 20 | Ethocel 45 | Ethocel 100 |
| 0.25 | 27 | 13 | 9 | 4 |
| 0.5 | 35 | 22 | 13 | 6 |
| 1 | 42 | 30 | 19 | 7 |
| 2 | 45 | 36 | 24 | 11 |
| 3 | 45 | 41 | 28 | 14 |
| 4 | 45 | 41 | 31 | 16 |
| 6 | 45 | 43 | 35 | 20 |
| 8 | 45 | 44 | 37 | 23 |
| 12 | 45 | 45 | 40 | 27 |

| | Cumulative dextromethorphan | | | |
|---|---|---|---|---|
| | released (mg) from uncoated | | | |
| | and Ethocel 100-coated resin complexes | | | |
| Time (hours) | No coating | 30% coating | 50% coating | 70% coating |
| 0.25 | 27 | 11 | 6 | 4 |
| 0.5 | 35 | 17 | 9 | 6 |
| 1 | 42 | 25 | 14 | 8 |
| 2 | 45 | 34 | 21 | 12 |
| 3 | 45 | 39 | 25 | 15 |
| 4 | 45 | 41 | 29 | 17 |
| 6 | 45 | 44 | 34 | 21 |
| 8 | 45 | 44 | 37 | 24 |
| 12 | 45 | 45 | 40 | 28 |

### Example 8

### Polymer-Coated Dextromethorphan/Resin Complexes by Fluid-Bed Coating

The dextromethorphan-loaded resin complex particles were coated with Aquacoat® ECD, Surelease®, and Kollicoat SR® 30D in a fluidized-bed coater, MFL-01 (Vector Corporation, Marion, Iowa) to obtain a predetermined weight gain. A bottom spray coating method (Wurster process) was applied for this process. The coating solution was diluted to 10.0 w/w % solid content. In order to enhance the film formation and the flexibility of the films, plasticizer (triethyl citrate) was added to Aquacoat® ECD and Kollicoat SR® 30D. In the case of Surelease®, no plasticizers are added. Formulations of the coating solution and operating conditions of the fluid-bed coater are shown in the table.

| | | | Kollicoat |
|---|---|---|---|
| | | | SR ® |
| Conditions | Aquacoat ® ECD | Surelease ® | 30D |
| Operating conditions | | | |
| Inlet air flow (l/m) | 45.0 | 43.0 | 42.0 |
| Inlet air temperature (° C.) | 80.0 | 80.0 | 60.0 |
| Exhaust temperature (° C.) | 27.5 | 26.7 | 24.3 |
| Nozzle pressure (psi) | 15.8 | 15.6 | 15.7 |
| Pump speed (rpm) | 9.0 | 10.0 | 10.0 |
| Formulation | | | |
| Aquacoat ® ECD (g) | 24.23 | — | — |
| Surelease ® (g) | — | 40.0 | — |
| HPMC (g) | 0.93 | — | — |
| Kollicoat SR ® 30D (g) | — | — | 70.0 |
| Triethyl citrate (g) | 1.80 | — | 1.1 |
| Water (g) | 73.04 | 60.0 | 149.5 |

### Example 9

### Release of Dextromethorphan from Polymer-Coated Resin Complex Particles

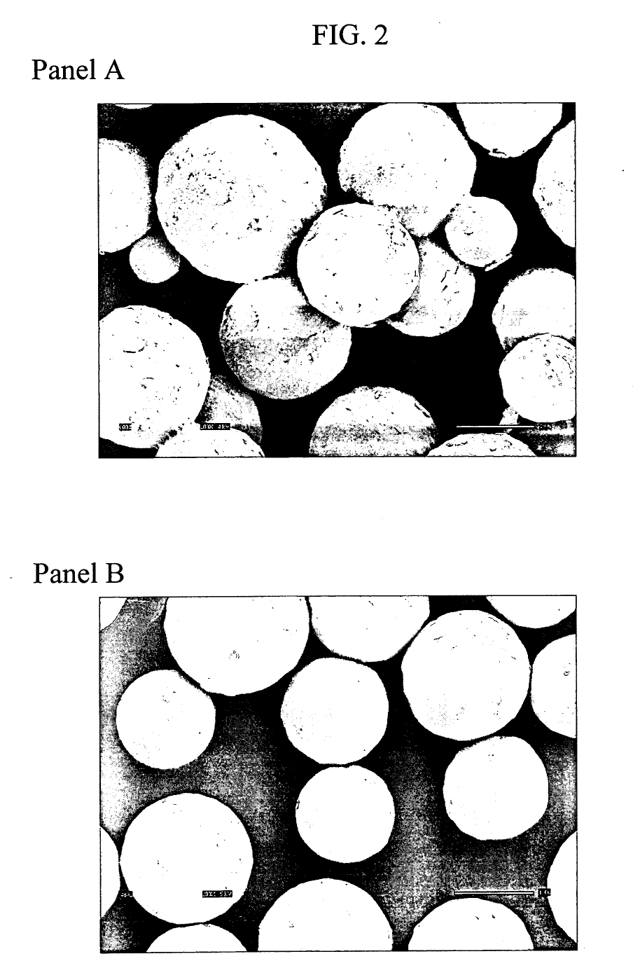

A drug release study from the polymer-coated dextromethorphan resin complex particles was conducted as described above. The following release data were obtained showing that the coating has significant sustained release properties and it depends on the different polymers and different level of coating. Moreover, FIG. 2 shows that there was no aggregation of the coated resin particles and average particle size was about 150 μm.

Cumulative
dextromethorphan
released (mg) from
Aquacoat ® ECD
coated resin complexes

| Time (hours) | 15% coating | 30% coating |
|---|---|---|
| 0.25 | 10 | 7 |
| 0.5 | 15 | 9 |
| 1 | 22 | 12 |
| 2 | 30 | 16 |
| 3 | 34 | 19 |
| 4 | 36 | 22 |
| 6 | 38 | 25 |
| 8 | 38 | 28 |
| 12 | 39 | 32 |

| | Cumulative dextromethorphan | | |
|---|---|---|---|
| | released (mg) | | |
| | from Surelease ® | | |
| | coated resin complexes | | |
| Time (hours) | 7.5% coating | 15% coating | 20% coating |
| 0.25 | 4 | 3 | 3 |
| 0.5 | 5 | 3 | 3 |
| 1 | 8 | 4 | 3 |
| 2 | 13 | 6 | 4 |
| 3 | 16 | 8 | 5 |
| 4 | 20 | 9 | 6 |
| 6 | 25 | 12 | 7 |
| 8 | 29 | 14 | 9 |
| 12 | 34 | 18 | 11 |

| | Cumulative dextromethorphan | | | |
|---|---|---|---|---|
| | released (mg) from | | | |
| | Kollicoat SR ® | | | |
| | 30D coated resin complexes | | | |
| Time (hours) | 5% coating | 10% coating | 15% coating | 20% coating |
| 0.25 | 7 | 4 | 4 | 3 |
| 0.5 | 11 | 6 | 5 | 4 |
| 1 | 17 | 8 | 7 | 5 |
| 2 | 25 | 14 | 10 | 7 |
| 3 | 31 | 18 | 14 | 9 |
| 4 | 34 | 21 | 16 | 11 |
| 6 | 38 | 27 | 21 | 14 |
| 8 | 41 | 31 | 25 | 17 |
| 12 | 43 | 37 | 32 | 22 |

### Example 10

### Fast-Melting Tablets using Microencapsulated Resin Complex Particles

Sustained release fast-melting tablets were prepared according to the following formulation. The amount of active ingredient was 60 mg in each tablet.

| Ingredient | Amount | |
|---|---|---|
| Coated drug/resin complex | 158.1 | mg |
| D-mannitol | 182.2 | mg |
| Maltodextrin | 45.7 | mg |
| Sucrose | 87.5 | mg |
| (used as a 70% aqueous solution) | | |
| Crospovidone | 15.0 | mg |
| Aspartame | 10.0 | mg |
| Sodium stearyl fumarate | 1.5 | mg |
| Total | 500.0 | mg/tablet |

Coated dextromethorphan/Dowex® 50WX4-400 complex particles (Example 2), D-mannitol, and maltodextrin were blended and then 70% sucrose solution was added dropwise. The obtained wet mass was passed through a #18 sieve, and the sieved wet granules were placed in a room at 20° C. and 25% relative humidity (RH) for 48 hours. Dried granules were passed through a # 18 sieve. The sieved granules were blended with crospovidone, aspartame, and lubricant (sodium stearyl fumarate). Blended granules of 500 mg were compressed on a compression punch with diameter of 12.6 mm at 300 lbs. When the compressed tablets were placed in the mouth, they melt in less than 60 sec and their release profiles are shown in the following table.

| | Cumulative dextromethorphan |
|---|---|
| | released (mg) from |
| Time (hours) | microencapsulated resin particles |
| 0.25 | 6 |
| 0.5 | 9 |
| 1 | 13 |
| 2 | 18 |
| 3 | 22 |
| 4 | 25 |
| 6 | 29 |
| 8 | 33 |
| 12 | 36 |

### Example 11

### Fast-Melting Tablets using Fluid-Bed Coated Resin Complex Particles

Sustained release fast-melting tablets were prepared according to the following formulation. The amount of the active ingredient was 60 mg in each tablet.

| Components | Amount (%) |
|---|---|
| Mannogem EZ Spray (mannitol) | 51.3 |
| Advantose FS 95 (Fructose) | 5.0 |
| Coated resin particles | 26.9 |
| Cherry flavor | 0.3 |
| Citric Acid | 0.5 |
| Aspartame | 2.0 |
| Sugar | 14.0 |

After the granules were prepared in the same way as the previous example, three different types of lubricants (magnesium stearate, PRUV®, and stearic acid 0.5% each) were added into the granules. For the lubricant blending, the pre-measured granules and lubricants were added into a bin blender. Blend time and speed were 15 min and 25 rpm, respectively. Tablets of 500 mg were compressed on a single punch Carver Laboratory Press (Carver Inc. Wabash, Ind.) at different compression pressures using plane-face punches with diameter of 0.5 inch and on a rotary tablet press (Manesty Betapress Model 13U18). The next table shows the release profiles depending on the different compression pressures.

Cumulative dextromethorphan released
(mg) from the fast-melting tablet with
different compression pressure

| Time (hours) | 200 lbs | 300 lbs | 500 lbs | 800 lbs |
|---|---|---|---|---|
| 0.25 | 2 | 3 | 3 | 3 |
| 0.5 | 4 | 4 | 4 | 5 |
| 1 | 6 | 6 | 6 | 7 |
| 2 | 9 | 9 | 10 | 11 |
| 3 | 13 | 12 | 14 | 14 |
| 4 | 15 | 15 | 17 | 18 |
| 6 | 20 | 20 | 22 | 23 |
| 8 | 24 | 24 | 26 | 27 |
| 12 | 31 | 29 | 32 | 33 |

### Example 12

### Diphenhydramine/Amberlite IRP-69 Complex

Diphenhydramine hydrochloride is a water-soluble antihistaminic drug. The diphenhydramine/Amberlite IRP-69 complex was prepared by a batch process. The purified ion-exchange resin (8 g) was suspended in a 5 w/v % of diphenhydramine hydrochloride aqueous solution (200 ml) under magnetic stirring at room temperature for 24 hours. The drug-loaded ion-exchange resin was separated from the supernatant by vacuum filtration, washed with de-ionized water to remove any uncomplexed drug, and then dried in an oven. The dried complexes were sieved through a #100 screen (150 micron).

### Example 3

### Fast-Melting Tablet Containing Diphenhydramine-IRP69 Complex Particles

Taste-masked, fast disintegrating tablets were prepared according to the following formulation. The diphenhydramine-IRP69 complex (105.3 mg) included diphenhydramine at a concentration equivalent to 50 mg of the free form.

| Ingredient | Amount | |
|---|---|---|
| Diphenhydramine-IRP69 complex | 105.3 | mg |
| D-mannitol | 240.0 | mg |
| Maltodextrin | 60.0 | mg |
| Sucrose | 68.2 | mg |
| (used as a 70% aqueous solution) | | |
| Crospovidone | 15.0 | mg |
| Aspartame | 10.0 | mg |
| Sodium stearyl fumarate | 1.5 | mg |
| Total | 500.0 | mg/tablet |

To the mixture of diphenhydramine-IRP69 complex, D-mannitol and maltodextrin previously blended, was gradually added 70% sucrose solution. It was preferred to add 0.2 ml aliquots of the sucrose solution while granulating with a hand mixer. The obtained wet mass was passed through a #18 sieve, and the sieved wet granules were placed in a room at 20° C. and 25% RH for 48 hours. Dried granules were passed through a #18 sieve. The sieved granules were blended with crospovidone and aspartame. The mixture was blended with lubricant, sodium stearyl fumarate. Lubricated granules of 350 mg were poured into a 0.5 inch die and subsequently compressed at 600 lb. When the compressed tablets were placed in the mouth, they melt in less than 30 seconds.

### Example 14

### Diphenhydramine/Amberlite IRP-64 Resin Complex

The diphenhydramine/Amberlite IRP-64 complex was prepared by a batch process. Diphenhydramine hydrochloride (14.04 g) was dissolved in a solution of sodium hydroxide (1.92 g) in de-ionized water (300 ml). The purified ion-exchange resin (10 g) was suspended in this solution under magnetic stirring at room temperature for 24 hours. The drug-loaded ion-exchange resin was separated from the supernatant by vacuum filtration, washed with deionized water to remove any uncomplexed drug, and then dried in an oven. The dried complexes were sieved through a #100 screen (150 micron).

### Example 15

### Fast-Melting Tablet Containing Diphenhydramine-IRP64 Complex

Taste-masked, fast disintegrating tablets were prepared according to the following formulation. The diphenhydramine-IRP64 complex (93 mg) included diphenhydramine at a concentration equivalent to 50 mg of the free form.

| Ingredient | Amount | |
|---|---|---|
| Diphenhydramine-IRP64 complex | 93.0 | mg |
| D-mannitol | 256.5 | mg |
| Maltodextrin | 64.1 | mg |
| Sucrose | 59.9 | mg |
| (used as a 70% aqueous solution) | | |
| Crospovidone | 15.0 | mg |
| Aspartame | 10.0 | mg |
| Sodium stearyl fumarate | 1.5 | mg |
| Total | 500.0 | mg/tablet |

To the mixture of diphenhydramine-IRP64 complex, D-mannitol and maltodextrin previously blended, was gradually added 70% sucrose solution. It was preferred to add 0.2 ml aliquots of the sucrose solution while granulating with a hand mixer. The obtained wet mass was passed through a #18 sieve, and the sieved wet granules were placed in a room at 20° C. and 25% RH for 48 hours. Dried granules were passed through a #18 sieve. The sieved granules were blended with crospovidone and aspartame. The mixture was blended with lubricant, sodium stearyl fumarate. Lubricated granules of 350 mg were poured into a 0.5 inch die and subsequently compressed at 600 lbs. When the compressed tablets were placed in the mouth, they melt in less than 30 sec.

### Example 16

### Cetirizine/Amberlite IRP-64 Resin Complex

Cetirizine hydrochloride is a water-soluble anti-histaminic drug. The cetirizine/Amberlite IRP-64 complex was prepared by a batch process. Cetirizine hydrochloride (2.4 g) was dissolved in a solution of sodium hydroxide (0.4 g) in de-ionized water (120 ml). Then, the pH of solution was adjusted to alkaline condition using sodium hydroxide. The purified ion-exchange resin (6 g) was suspended in this solution under magnetic stirring at room temperature for 24 hours. The drug-loaded ion-exchange resin was separated from the supernatant by vacuum filtration, washed with de-ionized water to remove any uncomplexed drug, and then dried in an oven. The dried complexes were sieved through a #100 screen (150 micron).

### Example 17

### Fast-Melting Tablet Containing Cetirizine/IRP64 Resin Complex

Taste-masked, fast disintegrating tablets were prepared according to the following formulation. The cetirizine/IRP64 complex (40 mg) included cetirizine at a concentration equivalent to 10 mg of the free form.

| Ingredient | Amount | |
|---|---|---|
| Cetirizine-IRP64 complex | 40.00 | mg |
| D-mannitol | 188.36 | mg |
| Maltodextrin | 47.09 | mg |
| Sucrose | 56.00 | mg |
| (used as a 70% aqueous solution) | | |
| Crospovidone | 10.50 | mg |
| Aspartame | 7.00 | mg |
| Sodium stearyl fumarate | 1.05 | mg |
| Total | 350.00 | mg/tablet |

To the mixture of cetirizine/IRP64 complex, D-mannitol and maltodextrin previously blended, was gradually added 70% sucrose solution. It was preferred to add 0.2 ml aliquots of the sucrose solution while granulating with a hand mixer. The obtained wet mass was passed through a #18 sieve, and the sieved wet granules were placed in a room at 20° C. and 25% RH for 48 hours. Dried granules were passed through a #18 sieve. The sieved granules were blended with crospovidone and aspartame. The mixture was blended with lubricant, sodium stearyl fumarate. Lubricated granules of 350 mg were poured into a 0.5 inch die and subsequently compressed at 600 lbs. When the compressed tablets were placed in the mouth, they melt in less than 30 sec.

### Example 18

### Drug Release Profiles from Fast-Melting Tablet Containing Cetirizine/IRP-64

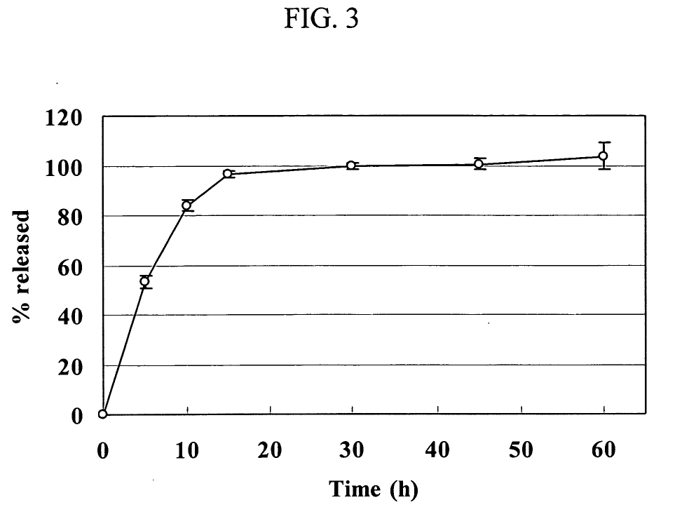

The drug release study from the fast-melting tablet containing the cetirizine/IRP-64 resin complexes was conducted according to USP 27 Apparatus 2 guidelines (paddle method) (Vankel® VK 7000, Vankel, Edison, N.J.) with 900 ml dissolution medium maintained at 37±0.5° C. and mixed at 100 rpm. The dissolution media used in this study were simulated gastric fluid (pH=1.2). The results are shown in FIG. 3.

### Example 19

### Fast-Melting Tablet Containing Cetirizine/Amberlite IRP-64 Resin Complex

The cetirizine/Amberlite IRP-64 complex was synthesized, purified, dried and sieved as described in Example 16. Taste-masked, fast disintegrating tablets were prepared according to the following formulation. The cetirizine/IRP64 complex (18 mg) included cetirizine at a concentration equivalent to 5 mg of the free form.

| Ingredient | Amount | |
|---|---|---|
| Cetirizine-IRP64 complex | 18 | mg |
| D-mannitol | 184.5 | mg |
| Advantose ™ FS 95 | 12.5 | mg |
| Sucrose | 35 | mg |
| (used as a 70% aqueous solution) | | |
| Magnesium Stearate | 1.25 | mg |
| Total | 251.25 | mg/tablet |

To the mixture of cetirizine/IRP64 complex, D-mannitol and Advantose™ FS 95 (co-dried system of fructose and starch from SPI Pharma (New Castle, Del.)) were mixed in a 6L granulation bowl. This mixture was granulated with 70% sucrose solution in Diosna mixer p1/6. The wet granules were then sieved through a #16 sieve and placed in a drying room overnight. The dry granules were then sieved through a #30 sieve. The above Cetirizine granules were blended with 1.0 g (0.5%) of magnesium stearate and tabletting was performed in a Manesty Betapress using two ⅜ inch punches to produce 251.25 mg cetirizine tablets. These tablets disintegrate in 20 seconds as tested by a volunteer. The friability of the tablets was 0.963% and the average hardness was 3.77 kP. The taste was tested by four volunteers. No or little bitter taste was found and there was no aftertaste after the tablet is swallowed.

### Example 20

### Fast-Melting Tablet Containing Cetirizine/Amberlite IRP-64 Complex

Cetirizine/Amberlite IRP-64 complex was synthesized, purified, dried and sieved as described in Example 16. Taste-masked, fast disintegrating tablets were prepared according to the following formulation. The cetirizine/IRP64 complex (18 mg) included cetirizine at a concentration equivalent to 5 mg of the free form.

| Ingredient | Amount | |
|---|---|---|
| Cetirizine-IRP64 complex | 18 | mg |
| D-mannitol | 184.5 | mg |
| Advantose ™ FS 95 | 12.5 | mg |
| Crospovidone | 5.0 | mg |
| Sucrose | 35.0 | mg |
| (used as a 70% aqueous solution) | | |
| Magnesium Stearate | 1.25 | mg |
| Total | 256.25 | mg/tablet |

To the mixture of cetirizine/IRP64 complex, D-mannitol and Advantose™ FS 95 were mixed in a 6L granulation bowl. This mixture was granulated with 70% sucrose solution in a Diosna mixer p1/6. The wet granules were then sieved through a #16 sieve. They were placed in a drying room overnight and the dry granules were then sieved through the #30 sieve. The cetirizine granules were blended with 1.0 g (0.5%) of magnesium stearate and 2% of crospovidone, and tabletting was performed in a Manesty Betapress using two ⅜ inch punches to produce 256.25 mg cetirizine tablets. These tablets disintegrated in 32 seconds as tested by a volunteer. The friability of the tablets was 0.402% and the average hardness was 3.46 kP. The taste was tested by four volunteers. Little or no bitter taste was noticed and there was no aftertaste when the tablet was swallowed.

### Example 21

### Fast-Melting Tablet Containing Cetirizine/Amberlite IRP-64 Complex

Cetirizine/Amberlite IRP-64 complex was synthesized, purified, dried and sieved as in Example 16. Taste-masked, fast disintegrating tablets were prepared according to the following formulation. The cetirizine/IRP64 complex (18 mg) included cetirizine at a concentration equivalent to 5 mg of the free form.

| Ingredient | Amount | |
|---|---|---|
| Cetirizine-IRP64 complex | 18 | mg |
| D-mannitol | 144 | mg |
| Advantose ™ FS 95 | 10 | mg |
| Sucrose (70% aqueous solution) | 28.0 | mg |
| Total | 200 | mg/tablet |

To a mixture of cetirizine/IRP64 complex, D-mannitol and Advantose™ FS 95 were mixed in a 6L granulation bowl. This mixture was granulated with 70% sucrose solution in Diosna mixer p1/6. The wet granules were then sieved through a #16 sieve and placed in the drying room overnight. The dry granules were then sieved through the #30 sieve. The above cetirizine granules were tabletted in a Manesty Betapress using two ⅜ inch punches to produce 200 mg Cetirizine tablets. These tablets disintegrated in 16 seconds as tested by a volunteer. The average hardness was 1.4 kP. The taste was examined by four volunteers. No or little bitter taste was claimed and there was no aftertaste when the tablet was swallowed.

The present invention has been described with reference to examples for purposes of clarity and understanding. It should be understood that certain modifications can be practiced within the scope of the appended claims and equivalent embodiments thereof.

What is claimed is:

1. A fast-melting tablet containing a plurality of compressed granules, each comprising an effective amount of particles of at least one active ingredient/ion-exchange resin complex, a dry binder, and a bulk diluent, wherein the active ingredient/ion-exchange resin complex comprises an active ingredient ionically bound to an ion-exchange resin.

2. The tablet of claim 1, further comprising a substance coating or microencapsulating the particles of active ingredient/ion-exchange resin complex.

3. The tablet of claim 1, wherein the active ingredient is an acidic, basic, or amphoteric pharmaceutical, nutritional, vitamin, mineral or dietary supplement.

4. The tablet of claim 3, wherein the active ingredient is an active pharmaceutical ingredient.

5. The tablet of claim 4, wherein the active ingredient is a free form of a basic pharmaceutical or a salt with a pharmaceutically allowed acid.

6. The tablet of claim 5, wherein the active ingredient is selected from diphenhydramine hydrochloride, cetirizine hydrochloride, dextromethorphan hydrobromide, and venlafaxine hydrochloride.

7. The tablet of claim 1, wherein the ion-exchange resin is a weakly or strongly acidic type, and mixtures thereof.

8. The tablet of claim 7, wherein an average diameter of particles of the active-ingredient/ion-exchange resin complex is from 10 to 400 μm.

9. The tablet of claim 1, wherein the weight ratio between the at least one active ingredient and the ion exchange resin is from 0.5:1 to 3:1.

10. The tablet of claim 2, wherein the coating substance is selected from vinyl polymers, (meth)acrylate polymers, cellulosic polymers, waxes, polysaccharides, and mixtures thereof.

11. The tablet of claim 10, wherein the vinyl polymer is selected from polyvinyl acetate and polyvinyl alcohol-polyethylene glycol.

12. The tablet of claim 10, wherein the cellulosic polymer is selected from ethylcellulose (EC), hydroxypropyl cellulose (HPC), hydroxypropylmethyl cellulose (HPMC), hydroxypropyl-methylcellulose phthalate (HPMCP), and cellulose acetate phthalate (CAP).

13. The tablet of claim 10, wherein the (meth)acrylate polymer is selected from aminoalkyl methacrylate copolymers, ammonioalkyl methacrylate copolymers, methacrylate copolymers, ethyl acrylate-methyl methacrylate coppolymer, metacrylic acid-ethyl acrylate copolymer, and mixtures thereof.

14. The tablet of claim 10, wherein the wax is selected from glyceryl behenate, polyethylene glycols, stearic acid, glyceryl monostearate, hydrogenated vegetable oils, and mixtures thereof.

15. The tablet of claim 10, wherein the polysaccharide is maltodextrin.

16. The tablet of claim 1, wherein the dry binder is selected from maltodextrin, dextrin, ethylcellulose, polymethacrylates, and pregelatinated starch, and mixtures thereof.

17. The tablet of claim 1, wherein the bulk diluent is selected from dextrates, dextrin, dextrose, fructose, lactitol, lactose, maltitol, maltose, mannitol, sorbitol, sucrose, erythritol, xylitol, microcrystalline cellulose, silicified microcrystalline cellulose, powdered cellulose, cellulose acetate, calcium sulfate, calcium carbonate, dibasic calcium phosphate, tribasic calcium phosphate, carboxymethylcellulose-calcium salt, and mixtures thereof.

18. The tablet of claim 1, further comprising a wet binder.

19. The tablet of claim 1, further comprising a natural or artificial sweetener, flavoring agent, or colorant.

20. A method of making a fast-melting tablet having taste-masking properties comprising:
providing a plurality of particles of an active ingredient/ion-exchange resin complex;
combining a dry binder and a bulk diluent with the resin complex particles to form an admixture thereof;
treating the admixture with an aqueous wet granulation solution effective to form a wet mass of agglomerated particles;
sieving and drying the agglomerated particles to isolate highly plastic granules; and
compressing the granules under low pressure to afford the fast-melting tablet.

21. The method of claim 20, further comprising applying a coating substance to the resin complex particles prior to combining with dry binder and bulk diluent.

22. The method of claim 21, wherein the coating substance is selected from vinyl polymers, (meth)acrylate polymers, cellulosic polymers, waxes, polysaccharides, and mixtures thereof.

23. The method of claim 20, wherein the active ingredient is an acidic, basic, or amphoteric pharmaceutical, nutritional, vitamin, mineral or dietary supplement.

24. The method of claim 23, wherein the active ingredient is an active pharmaceutical ingredient.

25. The method of claim 20, wherein the dry binder is selected from maltodextrin, dextrin, ethylcellulose, polymethacrylates, and pregelatinated starch, and mixtures thereof.

26. The method of claim 20, wherein the bulk diluent is selected from dextrates, dextrin, dextrose, fructose, lactitol, lactose, maltitol, maltose, mannitol, sorbitol, sucrose, erythritol, xylitol, microcrystalline cellulose, silicified microcrystalline cellulose, powdered cellulose, cellulose acetate, calcium sulfate, calcium carbonate, dibasic calcium phosphate, tribasic calcium phosphate, carboxymethylcellulose-calcium salt, and mixtures thereof.

27. The method of claim 20, wherein the wet granulation solution comprises at least one of sorbitol, acacia, alginic acid, carbomer, carboxymethylcellulose, cellulose, dextrin, gelatin, hydroxyethylcellulose, hydroxypropylcellulose, hydroxypropyl methylcellulose, methylcellulose, polydextrose, poly(ethylene oxide), povidone, sodium alginate, and mixtures thereof.

28. The method of claim 20, further comprising admixing the dried highly plastic granules with a superdisintegrant, superporous hydrogel, effervescent agent, lubricant, flavoring agent, or coloring agent prior to compressing.

29. The method of claim 28, wherein lubricant is dry-sprayed onto tablet tooling during compressing.

30. A fast-melting tablet prepared by the method of claim 20.
